(19) United States
(12) Patent Application Publication
(10) Pub. No.: US 2026/0114774 A1
(43) Pub. Date: Apr. 30, 2026

(54) SYSTEM FOR MEASURING CONTRACTIONS OF THE PELVIC FLOOR MUSCLES OF A PATIENT, AND METHOD FOR MEASURING CONTRACTIONS OF THE PELVIC FLOOR MUSCLES USING SUCH A SYSTEM

(71) Applicant: X6 INNOVATIONS, PARIS (FR)

(72) Inventors: Louise AUMONT, PARIS (FR); Erica Taylor PERRIER, PARIS (FR)

(21) Appl. No.: 18/837,854

(22) PCT Filed: Feb. 13, 2023
(86) PCT No.: PCT/EP2023/053516

§ 371 Date: Aug. 12, 2024

(30) Foreign Application Priority Data
Feb. 14, 2022 (FR) FR2201246

(51) Int. Cl. A61B 5/22; A61B 5/00; A61B 5/03; A61B 5/11

(52) U.S. Cl. CPC A61B 5/227; A61B 5/036; A61B 5/1107; A61B 5/4337; A61B 5/742; A61B 2562/0219; A61B 2562/0247

(57) ABSTRACT

System for measuring contractions of the pelvic floor muscles (PFM) of a patient, the system comprises a pelvic floor muscle probe configured to be positioned inside an internal canal of the patient, a data-processing module configured to compare a difference denoted ΔT between a pressure measurement time Tp and a position measurement time Tm and to compare the difference ΔT with a determined threshold value; in which the data-processing module is in communication with a notification device for notifying the patient that the synergy between the movement of the body and the contraction of the pelvic floor muscles is suboptimal when the difference between the pressure measurement time Tp and the position measurement time Tm is lower than or equal to the threshold value.

### FIELD OF THE INVENTION

The present invention relates to a system for performing exercises to strengthen the pelvic floor muscles of a patient.

More specifically, the invention relates to such a system making it possible to monitor whether Kegel exercises are being performed correctly by the patient.

### TECHNOLOGICAL BACKGROUND

The pelvic floor muscles (PFM) are a set of muscles which extend from the coccyx to the pubic bone and which, when they are contracted, make it possible to support the internal organs of the pelvis (in particular the bladder, the rectum and the anus) in order to ensure that the sphincters of the anus and the bladder are closed and to guarantee continence. They therefore play an important role in the control of urine and stool. The pelvic floor muscles also play an important role in sexual function.

However, following pregnancy, childbirth, trauma or even with age, the pelvic floor muscles can slacken, impacting the comfort and health of the patient. Several pathological conditions, such as cystocele (sagging of part of the bladder through the vaginal wall), urinary incontinence, rectocele (sagging of part of the rectum into the vagina), uterine prolapse (bulging of the uterus into the vagina), and bladder and sexual dysfunction, can be caused by a weakened state of the pelvic floor muscles.

Exercises and instruments have therefore been developed for strengthening the pelvic floor muscles. The exercises generally consist of series of contractions and relaxations of these muscles, to be performed consciously by the patient, possibly with the aid of a health professional, which make it possible to strengthen the pelvic floor muscles. These exercises are commonly called Kegel exercises. However, it is not enough to perform such exercises without monitoring their impact on the pelvic floor muscles. In fact, it needs to be possible to determine whether or not an exercise is being performed correctly, i.e. to verify whether the muscle or the group of muscles targeted by the exercise are being used correctly, at the risk not only of failing to achieve the desired strengthening, but also of damaging the pelvic floor muscles further.

In order to verify that the progress of the Kegel exercises is good, they are generally performed under supervision by a health professional, who manually and/or visually determines whether the PFMs are contracting correctly according to the instructions that they might give. However, manual supervision can be incomplete. Additionally, intervention by a professional makes it difficult to perform these exercises frequently and regularly, over the long term, as well as possibly causing embarrassment for the patient.

There are therefore devices making it possible to monitor Kegel exercises and detect when they are being performed wrongly, either by a professional or by the patient themselves, at home.

The general principle of these devices is to detect the contractions of the pelvic floor muscles by measuring, by means of a probe inserted in an internal canal (vaginal canal or anus) of the patient, the muscular activity or the pressure generated by the pelvic floor muscles, and to verify that the time of the contraction corresponds to the instruction given for the exercise. The intensity of the contractions can also be measured. A visualization of the contractions then makes it possible for the patient and/or for the professional to see the quality of the contractions.

The document CN110538432 describes an example of such a device making it possible to measure a pressure of the pelvic floor muscles in a relaxed state and in a contracted state. By comparing the value of the pressure in these two states, it then becomes possible to obtain an evaluation of the level of contraction of the pelvic floor muscles.

The document WO2017/118461 describes an example of a device with a display system making it possible to visualize the contractions. Such a system comprises pressure sensors, which measure the pressure exerted on the device, and a device making it possible to show these measurements visually as deformations of the device.

It is also known to propose visualizing the contractions using a display of the video game type, in which the contractions of the pelvic floor muscle control interactions or behaviours in the video game, for example on a smartphone. The document WO2021/144270 describes an example of such a display.

During a pelvic floor muscle strengthening exercise, contractions of muscles other than those of the pelvic floor can occur, consciously or unconsciously. To be precise, this concerns the abdominal girdle muscles, causing an increase in intra-abdominal pressure, which in turn increases the pressure exerted on the pelvic floor.

The document WO2021/144270 proposes measuring two pressures, which are distinct from each other, in the vaginal canal, at two different depths in the internal canal of the patient, namely a pressure in an area of the canal called the medial area and a pressure in an area of the canal called the distal area. These two measurements at two different depths make it possible, by comparing them, to determine whether and to what extent the abdominal pressure has been increased and therefore that the pelvic floor muscle strengthening exercise has not been performed correctly. This additional information makes it possible to improve the evaluation of the pelvic floor muscle strengthening exercises and to implement suitable corrections.

As a variant, it is known to detect a wrong contraction during a pelvic floor muscle strengthening exercise by following the movements of the device. The document WO2016/119002, for example, proposes using a gyroscope, possibly combined with an accelerometer, in an intravaginal device, in order to determine whether, during the performance of an exercise, the movement of the muscles corresponds well to a movement upwards. More specifically, the intravaginal device indicates the orientation of the probe with respect to a vertical reference direction in the vaginal canal in order to deduce therefrom whether the movement is correct. Data about the position of the patient can also be used to sort the results.

However, in real-life situations, dysfunctions of the pelvic floor muscles can arise during movements of the patient, for example in changing from a seated position to a standing position, and vice versa. In fact, the movements of a patient can exert a stress on the pelvic floor and lead to embarrassment, such as leakage of urine or stool, in particular if the pelvic floor muscles are not satisfactorily fulfilling their function.

There is therefore a need for a new system for measuring the contractions of the pelvic floor muscles which makes it possible to verify, while exercise is being performed, the effectiveness of the contractions of the pelvic floor muscles during a movement, in order to better assist the patient in performing the pelvic floor muscle strengthening exercises.

A first subject of the invention is to improve the quality of the pelvic floor muscle strengthening exercises.

A second subject of the invention is to match the movements of a patient to the contractions of the pelvic floor muscles.

A third subject of the invention is to make it easier for the patient to perform pelvic floor muscle strengthening exercise by themselves, without the aid of a health professional.

A fourth subject of the invention is to guarantee the comfort of the patient during the pelvic floor muscle strengthening exercises.

A fifth subject of the invention is to make it easier to evaluate the quality of the pelvic floor muscle strengthening exercises.

### SUMMARY OF THE INVENTION

Thus, according to a first aspect, the invention relates to a system for measuring contractions of the pelvic floor muscles of a patient. The system comprises in particular:
a pelvic floor muscle probe configured to be positioned inside an internal canal, in particular the vaginal canal or the anus and the rectum, of the patient and in contact with the internal surface of said canal. The probe comprises a body, an external surface, at least one pressure sensor suitable for measuring the pressure applied by the internal surface of the canal on the external surface of the probe and at least one movement sensor suitable for detecting and/or measuring a movement of the probe in a reference frame external to the probe,
a pressure-measuring module configured to calculate at least one differential pressure, denoted ΔP, in the internal canal of the patient on the basis of a pressure measurement. The differential pressure ΔP has, as reference pressure, values measured in a relaxed state of the patient in which the pelvic floor muscles are relaxed. The measurement module is configured to associate at least one measured differential pressure ΔP with a pressure measurement time Tp. In particular when the pressure measurement can be interpreted as a contraction of the pelvic floor muscles, the measurement module can associate a first time Tp1 with the initiation of a contraction and a second time Tp2 with the end of the same contraction;
a movement-measuring module configured to detect at least one movement of the body of the patient on the basis of a measurement of the movement of the probe and configured to associate the detected movement with at least one measurement time Tm. In particular when the movement measurement can be interpreted as a given set of movements of the body of the patient, the measurement module can associate a first time Tm1 with the start of a set of movements and a second time Tm2 with the end of the same set of movements;
a data-processing module in communication with the pressure-measuring module and the movement-measuring module. The data-processing module is configured to calculate a difference denoted ΔT between the pressure measurement time Tp and the movement measurement time Tm. The data-processing module is moreover configured to verify that the difference ΔT respects a determined condition to be respected.

The data-processing module is in communication with a notification device for notifying the patient that the synergy between the contractions of the abdominal muscles and the pelvic floor muscles is suboptimal when the difference ΔT between the pressure measurement time Tp and the position measurement time Tm contravenes, i.e. does not respect, the determined condition to be respected.

The condition is to be understood here as any rule making it possible to temporally compare the pressure measurement time Tp with the movement measurement time Tm, in order to verify that a pelvic floor muscle strengthening exercise is being carried out well.

Owing to these provisions, the system makes it possible to bring the movements of the body of the patient, for example when they change from the standing position to the seated position, or when they walk or run, into concordance with the contractions of the pelvic floor muscles. In particular, the use of a reference point external to the patient and to the probe makes it possible to obtain dynamic information, i.e. about the movements carried out by the body of the patient. By introducing the supervision of this concordance into the monitoring of the pelvic floor muscle strengthening exercises, the quality of the results obtained regarding the problems linked to the pelvic floor muscles is improved. For example, the system can verify that the contraction of the muscles over the period defined by Tp1 and Tp2 is maintained for the set of movements performed over the period defined by Tm1 and Tm2.

Moreover, by placing the movement sensor in the probe, the patient has only one device to introduce and manipulate, limiting the discomfort and making it easier for the patient to use alone.

According to different aspects, it is possible to provide one and/or the other of the features below taken alone or in combination.

According to an embodiment, the movement sensor can comprise at least one accelerometer, which measures an acceleration of the probe with respect to gravitational acceleration. The movement-measuring module can then be configured to detect a movement of the body as a whole, in the reference frame external to the probe and to the patient, when the measured acceleration is greater than or equal to a determined value.

Thus, the accelerometer can easily be of a small size, in order to be integrated in the probe, can be easily found on the market, has a suitable measuring precision and limits the costs.

According to an embodiment, the probe can comprise at least two pressure sensors operating independently of each other, and having distinct detection areas which are not superimposed. The processing module can thus measure a differential pressure in each detection area.

The difference in differential pressure in the two detection areas makes it possible to obtain an additional item of information about the quality of the contractions during the performance of a pelvic floor muscle strengthening exercise and makes it possible to guide the patient in order to improve the performance of the exercise.

According to an embodiment, the processing module can be configured to calculate a first difference ΔT1 between a first pressure measurement time Tp1 and a first movement measurement time Tm1. A first condition to be respected is that the first difference ΔT1 is comprised within a determined time interval.

The term interval is to be understood here as having at least one defined limit, the other limit being able to be undefined, i.e. more or less equal to infinity.

Similarly, the processing module can be configured to calculate a second difference ΔT2 between a second pressure measurement time Tp2, subsequent to the first pressure measurement time Tp1, and a second movement measurement time Tm2, subsequent to the first movement measurement time Tm1. A second condition to be respected can then be that the second difference ΔT2 is comprised within a determined time interval.

By on the one hand configuring the pressure module so that the first pressure measurement time Tp1 corresponds to the initiation of a contraction and the second pressure measurement time Tp2 corresponds to the end of said contraction, and on the other hand configuring the movement module so that the first movement measurement time Tm1 corresponds to the start of a set of movements and the second movement measurement time Tm2 corresponds to the end of said set of movements, the system can then detect whether a contraction is held for the set of movements. If the first condition and/or the second condition to be respected are contravened, then the notification device can notify the patient that the synergy between the contractions of the abdominal muscles and the pelvic floor muscles is suboptimal.

According to an embodiment, the probe can comprise a first section intended to be placed in a proximal area of the internal canal of the patient and a second section intended to be placed in a distal area of the internal canal when the probe is inserted there. The movement sensor can thus be housed in the second section of the probe, such that a measurement axis corresponds to the axis of a distal area of the internal canal.

Thus, the detection reference frame of the movements substantially corresponds to the natural reference frame of the patient, namely up/down, front/back and left/right. It is thereby made easier to read the detection of the movements.

In this case, the first section can comprise at least one first pressure sensor suitable for measuring the pressure applied by the canal of the patient on the external surface of the body of the probe in a proximal area of the canal and the second section can comprise at least one second pressure sensor suitable for measuring the pressure applied by the canal of the patient on the external surface of the body of the probe in a distal area of the canal.

It is thus possible, by comparing the two differential pressures on the basis of the measurements by each pressure sensor, to detect a contraction of the intra-abdominal muscles which is unfavourable for the performance of a good pelvic floor muscle strengthening exercise, and to alert the patient to this.

According to an embodiment, the probe can be elastically flexible such that it can adopt a first position, called the initial position, in which the first section is inclined with respect to the second section by an angle of inclination with a determined value, called the rest value, and a second position, called the curved position, in which the angle of inclination deviates from the rest value by flexibility. For example, the value of the angle of inclination at rest is substantially equal to 162°, and the value of the angle of inclination when the probe is in the curved position deviates from the value at rest by a maximum of +/−30°.

The probe can thus follow any movements of the internal canal during the performance of the exercises, limiting the discomfort of the patient.

According to an embodiment, the notification device can comprise an interface making it possible to visually display data relating to contractions performed by the patient.

Thus, the patient has easy access to information relating to the exercises carried out, preferably during the exercise, in order to correct themselves. These data are displayed for example in the form of a video game, such that they are easily intelligible to the patient.

According to a second subject, the invention proposes a method for measuring contractions of the pelvic floor muscles (PFM) using a system such as presented above, the method comprising the following steps:
calculating at least one differential pressure in the canal of the patient, denoted ΔP, the differential pressure having, as reference pressure, values measured in a relaxed state of the patient in which the pelvic floor muscles are relaxed, and associating a measured differential pressure ΔP with a pressure measurement time Tp. In particular when the pressure measurement is interpreted as a contraction of the pelvic floor muscles, a first time Tp1 can be associated with the initiation of the contraction and a second time Tp2 can be associated with the end of the contraction;
detecting a movement of the body of the patient on the basis of a measurement of the movement of the probe, and associating the detected movement with a measurement time Tm. In particular when the movement measurement is interpreted as a given movement of the body of the patient, the measurement module associates a time Tm1 with the start of the movement and a time Tm2 with the end of the movement;
calculating a difference ΔT between the pressure measurement time Tp and the position measurement time Tm and comparing the difference ΔT with a determined condition;
notifying the patient that the synergy between the contractions of the abdominal muscles and the pelvic floor muscles is suboptimal when the difference between the pressure measurement time Tp and the position measurement time Tm contravenes, i.e. does not respect, the determined condition.

### BRIEF DESCRIPTION OF THE DRAWINGS

Embodiments of the invention will be described below with reference to the drawings, described briefly below:

FIG. 1 represents a diagram illustrating a system for measuring contractions of the pelvic floor muscles according to an embodiment according to the invention;

FIG. 2 represents a three-dimensional diagrammatic view of an embodiment of a pelvic floor muscle probe for the system of FIG. 1;

FIG. 3a and FIG. 3b each represent a diagrammatic example of curves obtained using the measurement system of FIG. 1.

In the drawings, identical references designate identical or similar objects.

### DETAILED DESCRIPTION

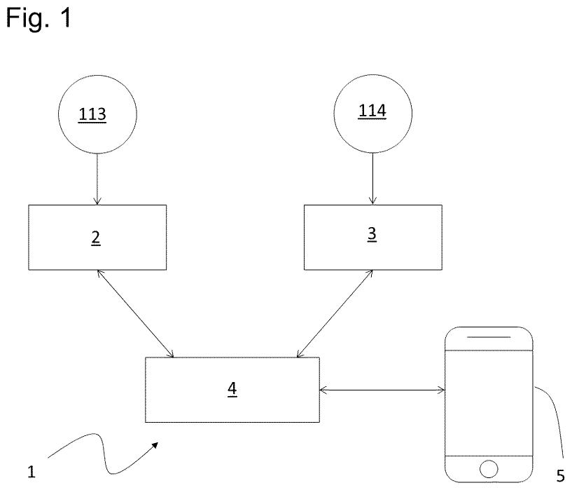

A system 1 for measuring contractions of the pelvic floor muscles, also denoted PFMs, is represented diagrammatically in FIG. 1. More specifically, such a system 1 can be used to follow the performance of Kegel exercises, which make it possible to strengthen the pelvic floor muscles, for personal use, at home, or for use aided by a health professional.

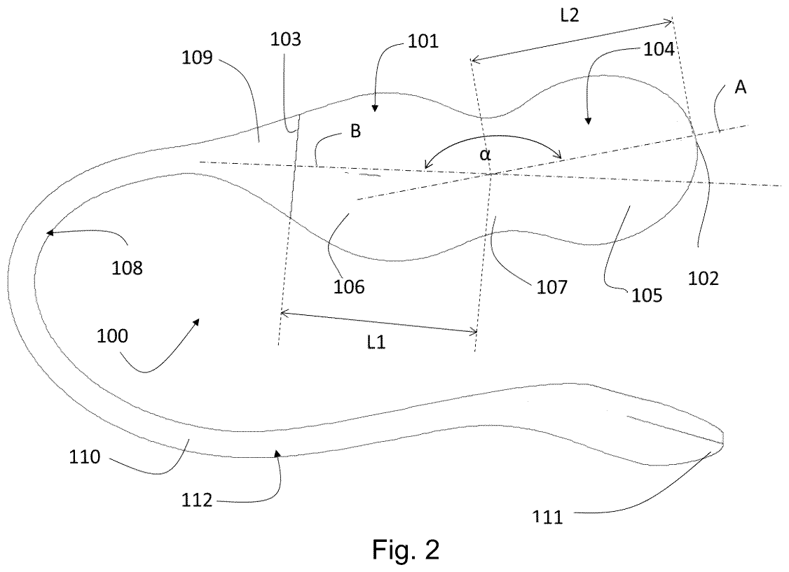

Such a system 1 comprises in particular a probe 100, illustrated more specifically in FIG. 2. The probe 100 is intended to be inserted at least partly in an internal canal, and in particular the vaginal canal or the anus of a patient.

In the following, the case where the internal canal is the vaginal canal is considered in particular. However, the system described is suitable for the probe to be inserted in the anus and the rectum for example.

The probe 100 comprises a body 101 extending substantially in a longitudinal direction between a proximal end 102 and a distal end 103.

The adjectives “distal” and “proximal” are here taken to refer to the position of the probe 100 when it has been inserted in the internal canal of the patient, the part which is towards the introitus being distal and the part which is towards the cervix being proximal.

The body 101 comprises an external surface 104, enveloping the body 101, made of biocompatible material, suitable for the anatomy of the human body in order to be able to be inserted in the vaginal canal, and made of liquid-tight material.

According to the embodiment illustrated in FIG. 2, the body 101 of the probe 100 comprises three sections. A first section 105 substantially in the shape of an oblong ball extends from the proximal end 102 and is separated from a second section 106 also in the shape of an oblong ball by a third section 107 of constriction, i.e. with a smaller diameter than that of the first section 105 and of the second section 106. The first section 105 and the second section 106 are substantially rotationally symmetric, but not necessarily.

The probe 100 moreover comprises a portion 108 called the tail portion, lengthening the second section 106 in the longitudinal direction. More specifically, the tail portion 108 comprises a junction section 109 fastened to the second section 106, on the proximal end 103, and a grip section 110 substantially in the form of a wire, extending to a free end 111, intended to be gripped by the patient. The junction section 109 ensures the continuity of shape between the second section 106 and the grip section 110. The first portion 105 and the second portion 106 are intended to be inserted inside the vaginal canal. The tail portion 108 is intended to be introduced partially inside the vaginal canal of the patient, at least part of the grip section 110 remaining outside. As a result, the tail portion 108 is also covered, at least over a portion corresponding to the one that will be introduced into the vaginal canal, and preferably over all of its surface, with an external surface 112 made of biocompatible material, suitable for the anatomy of the human body in order to be able to be inserted in the vaginal canal, and made of liquid-tight material. The tail portion 108 can be continuous with the body 101. For example, the external surface 104 of the body 101 is continuous with the external surface 112 of the tail portion 108, such that there is no discontinuity of surface. As a variant, the tail portion 108 can be added onto the body 101, for example assembled at the distal end 103 by any suitable means.

Thus, in other words, the sections 105 and 106 form two successive bulb-shaped domes, the undulated general shape of the probe 100 making it easier to insert it in the internal canal of the patient while limiting the embarrassment and discomfort once the probe is in place in the vaginal canal. The tail section 108, and more specifically the grip portion 111, remains available at least partially outside the canal in order to make it easier to pull the probe 100 out of the vaginal canal. More preferably, the tail section 108 is flexible, i.e. it deforms without breaking when it is manipulated manually by a user, at least in part, and preferably at least over the grip portion 111. When the probe 100 is inserted in the vaginal canal, the tail section 108 is thus easily put in a position which does not cause the patient discomfort when performing the exercises. The external surface 104 envelops at least the first section 105 and the second section 106, such that the probe 100 is in contact with the internal wall of the vaginal canal at least via a portion of the external surface 104 around the first section 105 and the second section 106.

The length L of the body 101 can be defined as being the sum of the length L1 of the distal section 106 and the length L2 of the proximal section 105. More specifically, the length L1 is, for example, measured between the distal end 102 and the centre of the constriction section 107, and the length L2 is, for example, measured between the centre of the constriction section 107 and the proximal end 103. According to an embodiment, the length L of the body 101 of the probe 100 is approximately 66 mm (millimetres), so as to correspond on average to two thirds of the length of the vaginal canal of a human patient. In practice, the probe 100 is dimensioned such that the length of the inserted part, possibly comprising part of the tail portion 108, in the vaginal canal corresponds approximately to 66 mm. Thus, the proximal end 102 of the probe 100 remains, from the opening of the vaginal canal, in the first two thirds of the length of the vaginal canal, remote from the last third of the length of the vaginal canal where the cervix is located, which is therefore a sensitive area. The comfort of the patient is thus ensured.

According to an embodiment, the body 101 of the probe 100 is elastically flexible, i.e. it can deform without breaking under the effect of a stress, and take its initial shape again in the absence of stress. For example, the body 101 is flexible at least at the level of the first constriction section 107, such that the first section 105 can be inclined with respect to the second section 106. Thus, the probe 100 can adopt a first position, called the initial position, corresponding to the position in the absence of stress, and a position called the curved position. The initial position can correspond to an inclination between the first section 105 and the second section 106 with an angle α of inclination with a determined value, called the rest value, suitable for the human anatomy. For example, when the probe 100 is intended to be inserted in the vaginal canal of a patient, it has been determined that a rest angle a of 162° is anatomically suitable. This angle α is measured between an axis A of extension of the first portion 105, which is for example the axis of rotation, and an axis B of extension of the second portion 106, which is again the axis of rotation for example. In order to correspond to the anatomy of the human body, the flexibility of the body 101 of the probe 100 makes it possible for the angle a of inclination, when the probe 100 adopts a curved position, to vary by +/−30° with respect to the value at rest.

By virtue of its shape, the probe 100, when it is inserted in the vaginal canal, substantially follows the curvature of the vaginal canal, possibly by using its flexibility properties, making it easier to insert and limiting the discomfort for the patient.

The probe 100 comprises at least one pressure sensor 113, which makes it possible to measure the pressure applied by the internal surface of the vaginal canal on the external surface 104 of the probe 100.

Pressure sensor here designates any sensor or set of sensors making it possible to gather information about the pressure.

Preferably, the probe can comprise two pressure sensors, namely a first sensor housed in the first section 105 and a second sensor housed in the second section 106. The two sensors operate independently of each other, i.e. the measurement performed by one is not influenced by the measurement performed by the other. Their detection areas are distinct from each other and do not overlap, i.e. each sensor in each case measures the pressure exerted by a muscle or a set of muscles on the probe which is different and distinct from the muscle or set of muscles exerting the pressure on the second sensor. Each sensor thus measures the pressure in a distinct area of the vaginal canal. By virtue of the position of the first section 105 and of the second section 106, a sensor placed in the first section 105 is intended to measure the pressure exerted on the probe 100 in an area of the vaginal canal called the proximal area, and the other sensor placed in the second section 106 is intended to measure the pressure exerted on the probe 100 in an area of the vaginal canal called the distal area. The presence of the first constriction area 107 contributes to the decoupling and to the separation between the measurements performed by the two sensors.

The probe 100 thus conforms to the probe described in the above-mentioned document WO2021/144270. More specifically, it makes it possible to measure two pressures, called differential pressures, taking into account a reference pressure measured in a relaxed state of the patient, when the pelvic floor muscles are in a relaxed or unflexed state. A comparison between these two differential pressures makes it possible in particular to detect contractions considered to be “wrong”. More specifically, it involves detecting an increase in intra-abdominal pressure.

In fact, contractions of the abdominal girdle muscles can occur during pelvic floor muscle strengthening exercises. Contraction of the abdominal girdle muscles beyond a certain level can imply that the pelvic floor muscle strengthening exercise is not being performed properly. Contraction of the abdominal girdle muscles can imply an increase in intra-abdominal pressure.

To this end, the measurement system 1 comprises a pressure-measuring module 2 which is in communication with at least one sensor 113, and in practice with the two pressure sensors of the probe in order to receive their measurements. In a prior step, the pressure-measuring module 2 recorded at least one reference value, so as to calculate at least one differential pressure denoted ΔP at a given moment. This differential pressure is then compared with a threshold pressure value in order to determine whether the contractions are sufficient depending on the exercise under consideration. In the case of two pressure sensors 113, the pressure-measuring module 2 can calculate two differential pressures denoted ΔP1 and ΔP2, which can each be compared with a threshold pressure value, and which can be compared with each other in order to detect any excessive increase in intra-abdominal pressure.

The threshold pressure value or values can be fixed, prerecorded in the pressure-measuring module 2, or be personalized, determined for example by a calibration step during a use of the probe 100 on the patient then recorded in the pressure-measuring module 2.

The measurement module 2 can be integrated in the probe 100 or be remote, the data of the pressure measurements from the sensors being transmitted to the pressure-measuring module 2 by a suitable transmission system, wired or not, or comprise a part integrated in the probe 2 and a remote part.

The probe 100 additionally comprises at least one movement sensor 114, making it possible to detect and/or measure a movement of the probe 100 in a reference frame external to the probe 100, and external to the patient. The function of the movement sensor 114 is thus to detect and/or measure movements of the body of the patient in said external reference frame, when the probe 100 is inserted in the vaginal canal of the patient. The movements detected and/or measured in this way by the movement sensor 114 do not correspond to internal contractions of the muscles of the patient, but rather to a displacement of all or part of the body of the patient in the external reference frame. A movement is thus to be understood in the dynamic sense of the term, as being for example a temporal succession of positions of the body of the patient in the external reference frame.

The movement sensor 114 comprises for example at least one accelerometer, which measures an acceleration of the probe 100 with respect to gravitational acceleration in three dimensions, i.e. according to an orthogonal reference point of dimension three. A movement is recorded for example by the movement sensor 114 as being a translation vector in the external reference frame. In the same way as previously, movement sensor here designates a single sensor or a set of sensors making it possible to detect a movement and/or to provide a measurement of the movement.

The movement sensor 114 is placed, for example, in the first section 105 or the second section 106 of the probe 100, which in each case offer sufficient volume to house it. In theory, however, the movement sensor 114 can be placed anywhere in the probe 100. According to an embodiment, the sensor 114, when it comprises an accelerometer, is placed in the distal section 106 of the body 101 of the probe 100. Thus, a measurement axis of the accelerometer is aligned with the distal area of the vaginal canal, a second measurement axis substantially corresponds to the left/right axis of the patient and the third measurement axis substantially corresponds to the front/back axis of the body of the patient. It is thereby made easier to read the detection of the movements, so as to also make corrections easier for the patient.

By placing the movement sensor 114 in the body 101 of the probe 100 with the pressure sensors 113, it is not necessary to multiply the devices on the patient, the probe 100 comprising all of the required instruments, limiting the discomfort for the patient.

The system 1 then comprises a movement-measuring module 3, in communication with the movement sensor 114, in order to detect a movement of the body of the patient. More specifically, the movement-measuring module 3 receives the measurement from the movement sensor 114. This measurement can correspond to a detection signal, of the type 0 in the case of absence of movement or 1 in the case of movement, such that the movement-measuring module 3 detects all movement of the patient without distinction. As a variant, the measurement of the movement by the sensor 114 can correspond to a value dependent on the amplitude of the movement and/or on the direction of the movement. In this case, the movement-measuring module 3 receives the measurement from the movement sensor 114 and compares it with a threshold movement value in order to determine whether the measurement of the movement has reached a sufficient value to consider that a significant movement has been performed. The threshold movement value can be fixed, prerecorded in the movement-measuring module 3. It can moreover be personalized, determined by a calibration step on the basis of one or more specific exercises performed by the patient when the probe 100 is inserted in the vaginal canal then recorded in the movement-measuring module 3. The threshold movement value can moreover depend on the envisaged exercise, such that several threshold values are recorded and/or determined by calibration, then the threshold value is chosen at the time of a given exercise. Thus, when the movement-measuring module 3 determines that the measured movement is greater than or equal to the corresponding threshold movement value, it considers that a movement has been detected. As another variant, or in combination, the measurement of the movement by the movement sensor 114 can be recorded by the measurement module 3.

The detected movement is preferably a translation in the reference frame external to the patient.

For example, the movement module 3 can detect a translation mainly along a vertical axis in the reference frame external to the probe and to the patient, indicating a movement such as changing from the seated position to the standing position and vice versa, or a translation mainly along a horizontal axis in the reference frame external to the probe and to the patient, indicating a movement such as walking, running, etc.

The system 1 makes it possible to link the contractions of the pelvic floor muscles to the movements of the patient in order to integrate the dynamic notion of movement in the pelvic floor muscle strengthening exercises and improve the quality of the exercises. To this end, the pressure-measuring module 2 is configured to associate a time Tp with each differential pressure measurement ΔP, when it is greater than a threshold value. Similarly, the movement-measuring module 3 is configured to associate a time Tm with each detected movement. The system 1 then comprises a data-processing module 4, which is in communication with the pressure-measuring module 2 and with the movement-measuring module 3. The data-processing module 4 is configured to receive the time data Tp and Tm from the two other modules 2, 3 and to calculate a difference ΔT between the two times Tp and Tm. The difference ΔT is then compared with a determined time condition in order to verify that the contraction and the movement are performed close enough to each other to validate the exercise. Thus, the determined condition on the difference ΔT can result from different situations depending on the needs of the patient in particular. For example, the condition can relate to ΔT not exceeding or not being lower than a threshold value, as will be explained below. ΔT can be expressed as the absolute value of the difference between Tp and Tm.

In fact, studies have shown that it was beneficial to take into account the movements of the patient during the performance of pelvic floor muscle strengthening exercises in order to verify that the contraction of certain pelvic floor muscles and the performance of a movement occur simultaneously. In the case of female anatomy, the synergy, i.e. the temporal concordance of the contractions, of the pelvic floor muscles and of the transverse abdominal muscle for example is observed more significantly in women who do not have symptoms of urine leakage than in women who do.

The condition on the difference ΔT can be fixed, prerecorded in a memory of the device 1 linked to the data-processing module 4. The condition can, as a variant, be personalized, determined by a calibration step on the basis of specific exercises performed by the patient with the probe then recorded in the data-processing module 4. It can moreover depend on the pelvic floor muscle strengthening exercise targeted. Thus, several conditions on the difference ΔT can be prerecorded or determined by calibration.

For example, when the difference ΔT is greater than or equal to a threshold time value, the system 1 can consider that the exercise is not being performed correctly, the pelvic floor muscles being contracted too early or too late with respect to the time of the movement.

The system can also determine that, for a given movement, the profile of the contractions does not correspond to an expected profile, for example when the maximum level of contraction is achieved at a time which does not coincide with the expected time during the performance of a movement.

The system 1 can also detect a relaxation of the pelvic floor muscles during a movement, when the movement has not been performed in full.

The data-processing module 4 is then in communication with a notification device 5 for notifying the patient that the synergy between the contractions of the abdominal muscles and the pelvic floor muscles is suboptimal when the difference between the pressure measurement time Tp and the position measurement time Tm contravenes the determined condition. The notification device 5 can then for example comprise in particular an interface for visually displaying data relating to the contractions performed for the patient and/or a health professional. It is for example a personal device of the smartphone type. Preferably, the notifications are displayed in real time, during the performance of the exercise or exercises.

The probe 100 can additionally comprise a translation sensor making it possible to measure the position of the probe 100 with respect to the patient, along the axis of the vaginal canal, which is used as reference. In practice, when the movement sensor 114 comprises an accelerometer, this same accelerometer can act as a translation sensor. The system 1 can then additionally comprise a warning module, which receives the position measurements from the translation sensor, and which can calculate a difference ΔL between the position of the probe 100 along the reference axis with a reference position along the reference curve. The warning module is for example a submodule of the movement-measuring module 3. A warning signal can then be generated, for example by the data-processing module 4, when the difference ΔL in position reaches a threshold warning value. The signal can be transmitted to the interface 5, in order to warn the patient that the probe 100 has shifted in an abnormal way. This warning makes it possible in particular to warn the patient and/or the health professional when the probe 100 has shifted so as to come out of the vaginal canal.

Each module 2, 3 and 4 can be integrated wholly or partially in the probe 100. They can be physically distinct from each other or, on the other hand, form part of one and the same computing system, which performs all of the functions described here. Each module 2, 3 and 4 can also be combined wholly or partially with another module.

The system 1 can thus be used to detect a potential problem in the synergy between the movement of the body and the contraction of the pelvic floor muscles. It can also be used to set up exercises targeted at working on this synergy. In particular, the notification device 5 can serve to display an evaluation of this synergy via a video game. For example, a video game character is controlled by the synergy, making the exercises fun and entertaining for the patient.

More specifically, from a start time of an exercise, the movement-measuring module 3 can record a plurality of movement measurements at regular intervals and associate each measurement with a time Tm in order to describe a set of movements of the patient. In fact, on the basis of the movement measurements, the processing module 4 can recognize a pattern recorded in a memory of the device 1 which corresponds to a type of exercise, for example a squat or a series of squats, a walk or a run, a movement for sitting down on or getting up from a chair, etc. At the same time, on the basis of the same start time for the exercise in question, the pressure-measuring module 2 can record a plurality of differential pressures ΔP at regular intervals, when they exceed a threshold value, and associate each one with a time Tp. The processing module 4 can then compare the temporal movement data and the temporal pressure data over a set of movements of the patient.

According to a first embodiment example, the processing module 4 can identify a time Tpmax for which the differential pressure measurement ΔP [is maximal and compare this time Tpmax with a time Tme corresponding to a predetermined movement for the planned exercise. For example, the time Tme can correspond to the movement time in which a translation along a vertical axis in the reference frame external to the patient reaches a particular value. According to this example, the processing module 4 can choose the condition on the difference ΔT as being expressed on the basis of a threshold value a in the following manner:

ΔT=|Tpmax−Tme|≤α

According to a second example, the processing module 4 can identify a first time Tp1 marking the initiation of a contraction as being the first occurrence, from the start time, of a differential pressure measurement ΔP greater than an associated threshold value and a second time Tp2 marking an end of the same contraction as being the first occurrence following a differential pressure measurement ΔP lower than the threshold value. It is then considered that the contraction is held between these two times Tp1 and Tp2. Similarly, the processing module 4 can identify a first time Tm1 marking a start of a set of movements and a second time Tm2 marking the end of the same set of movements, on the basis of the recorded patterns. The system 1 can thus verify that the contraction between Tp1 and Tp2 is maintained at least for the whole duration of the set of movements, i.e. over all of the period between Tm1 and Tm2.

For example, to obtain this verification, the processing module 4 can then express two conditions to be respected on the basis of four threshold values α11, α12, α21, α22 in the following manner:
A first condition:

α11≤ΔT1=Tp1−Tm1≤α12, where α11 is negative and α12 is positive or zero
A second condition:

α21≤ΔT2=Tp2−Tm2≤α22, where α21 is negative or zero and α22 is positive

If one of these two conditions, or both, is contravened, i.e. is not respected, then the data-processing module 4 sends the information to the notification device 5 to notify the patient that the exercise was not done correctly: either the contraction of the pelvic floor muscles started too late or it was relaxed too early, or both. The data-processing module 4 can distinguish between these situations.

Preferably, for a pelvic floor muscle strengthening exercise, it is acceptable that the contraction of the pelvic floor muscles is initiated much earlier than the start of the movement, whereas the acceptable period if it is initiated after the start of the movement is comparatively very short, or even zero. Thus, for example, the above threshold value α11 can be several seconds, whereas the threshold value α12 can be of the order of microseconds, or even zero. Similarly, it is acceptable that the contraction of the pelvic floor muscles is relaxed much later than the end of the movement, whereas the acceptable period if it is relaxed before the end of the movement is comparatively very short, or even zero. Thus, for example, the above threshold value α22 can be several seconds, whereas the threshold value α21 can be of the order of microseconds, or even zero.

According to a particular embodiment, α11 can be undefined, and α12 can be zero. The first condition expressed above can then be written as:

ΔT1=Tp1−Tm1≤0

Similarly, α21 can be zero and α22 can be undefined, such that the second condition expressed above can be written as:

0≤ΔT2=Tp2−Tm2

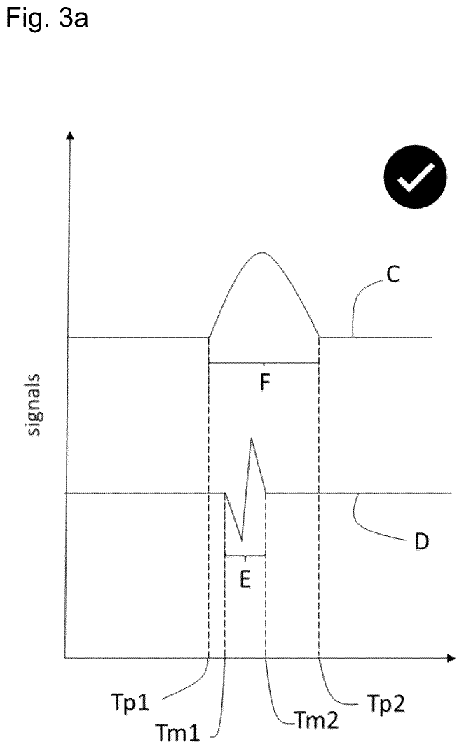

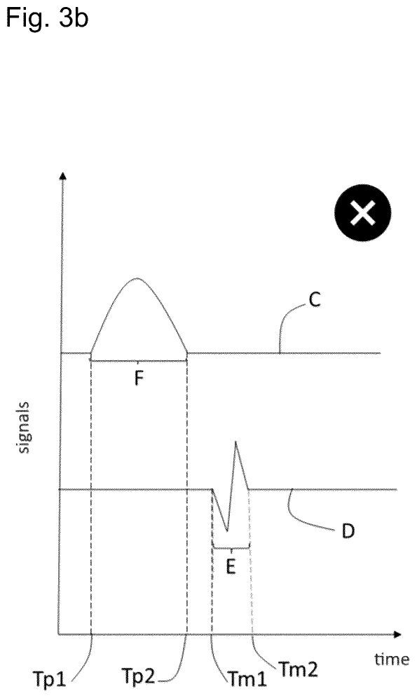

An illustrative example will now be presented using FIGS. 3a and 3b, in which the condition to be respected is that of the second example above: the contraction is maintained for the whole duration of the set of movements.

The patient is asked to perform an exercise in which they carry out a set of movements with their body, for example a squat. The exercise asked for implies a pattern in the set of the movement measurements of the body of the patient, which is particular to it and which is identifiable for example by the data-processing module 4. For the whole duration of the set of movements, the patient is supposed to contract the pelvic floor muscles, which is measured by the pressure module 2.

The curve C corresponding to the signal of the measurement by the pressure-measuring module 2 as a function of time and the curve D corresponding to the signal of the measurement by the movement-measuring module 3 as a function of time have been represented diagrammatically in FIGS. 3a and 3b. The exercise asked for is identifiable by its pattern over the period denoted E of the curve D. The movement-measuring module 3 then associates a time Tm1 with the start of the set of movements and a time Tm2 with the end of the same set of movements, which times enclose the portion E of the curve D. Similarly, the pressure-measuring module 2 associates a time Tp1 with the initiation of the contraction of the pelvic floor muscles and a time Tp2 with the end of the contraction of the pelvic floor muscles, which times enclose a period which is denoted F on the curve C and which corresponds to a contraction of the pelvic floor muscles. The data-processing module 4 can then compare Tm1, Tm2, Tp1 and Tp2.

In the example of FIG. 3a, Tp1 is smaller than or equal to Tm1 and Tm2 is smaller than or equal to Tp2, validating the exercise: a contraction of the pelvic floor muscles is maintained for the whole duration of performance of the movement of the body exerting a pressure on the pelvic floor in order to counter this pressure and to protect the pelvic floor muscles. There is no time interval comprised between Tm1 and Tm2 which is not also comprised between Tp1 and Tp2.

In the example of FIG. 3b, Tp1 is smaller than or equal to Tm1 but Tm2 is strictly greater than Tp2, the difference between Tm2 and Tp2 being greater than a threshold value, invalidating the exercise: there is a non-zero time interval comprised between Tm1 and Tm2 which is not comprised between Tp1 and Tp2, a contraction of the pelvic floor muscles is not maintained for the whole duration of the movement of the body exerting a pressure on the pelvic floor.

An aim of the measurement system 1 can be to verify that the contraction of the pelvic floor muscles is maintained for the whole duration of the movement of the body, i.e. for the whole duration of the exercise. In other words, the system 1 seeks to verify that the pressure exerted by the pelvic floor muscles on the probe 100 is maintained for the whole duration of the exercise.

An exercise will be considered incorrect when the contraction of the pelvic floor muscles is not held for the whole duration of the exercise, for example in the following cases:
The contraction is performed before the start of the set of movements, but it is not held for the whole duration of the exercise. In this case, the movement module 4 can detect that the measurement time Tm1 of the initiation of the set of movements, start of the period E, is effectively greater than or equal to the measurement time Tp1 of the initiation of the increase in pressure, characteristic of a contraction of the pelvic floor muscles, start of the period F, but that the measurement time Tm2 of the end of the same set of movements, end of the period E, is strictly greater than the measurement time Tp2 of the end of the contraction of the pelvic floor muscles, end of the period F.
The contraction is performed after the start of the set of movements. In this case, the movement module 4 can detect that the measurement time Tm1 of the initiation of the movement, start of the period E, is strictly smaller than the measurement time Tp1 of the initiation of the increase in pressure, characteristic of a contraction of the pelvic floor muscles, start of the period F.
No contraction is performed. In this case, no pressure measurement time Tp1 or Tp2 is taken into account by the processing module 4. There is therefore a time interval comprised between Tm1 and Tm2 during which no contraction of the pelvic floor muscles is performed, which is therefore not comprised between Tp1 and Tp2, since Tp1 and Tp2 do not exist.

Conversely, an exercise will be considered as done correctly when a contraction is performed before the start of a set of movements or simultaneously with the start of the set of movements, and is held until at least the end of the set of movements. In this case, the processing module 4 can verify that Tp1 is smaller than or equal to Tm1 and that Tm2 is smaller than or equal to Tp2, validating the exercise: a contraction of the pelvic floor muscles is maintained for the whole duration of performance of the movement of the body exerting a pressure on the pelvic floor in order to counter this pressure and protect the pelvic floor muscles. In other words again, there is no time interval comprised between Tm1 and Tm2 which is not also comprised between Tp1 and Tp2.

1. A system for measuring contractions of the pelvic floor muscles (PFM) of a patient, the system comprising:
A probe of the pelvic floor muscles configured to be positioned inside an internal canal of the patient and in contact with the internal surface of said canal, said probe comprising a body, an external surface, at least one pressure sensor suitable for measuring the pressure applied by the internal surface of the canal on the external surface of the probe and at least one movement sensor suitable for detecting and/or measuring a movement of the probe in a reference frame external to the probe and to the patient,
A pressure-measuring module configured to calculate at least one differential pressure, denoted ΔP, in the internal canal of the patient on the basis of a pressure measurement, the differential pressure ΔP having, as reference pressure, values measured in a relaxed state of the patient in which the pelvic floor muscles are relaxed, and the measurement module being configured to associate at least one measured differential pressure ΔP with at least one pressure measurement time Tp;
A movement-measuring module configured to detect at least one movement of the body of the patient on the basis of a measurement of the movement of the probe in a reference frame external to the probe and to the patient and configured to associate the detected movement with at least one measurement time Tm;
A data-processing module in communication with the pressure-measuring module and the movement-measuring module, the data-processing module being configured to calculate a difference denoted ΔT between the pressure measurement time Tp and the movement measurement time Tm, the data-processing module being moreover configured to verify that the difference ΔT respects at least one determined condition to be respected;
in which the data-processing module is in communication with a notification device for notifying the patient that the synergy between the contractions of the abdominal muscles and the pelvic floor muscles is suboptimal when the difference ΔT between the pressure measurement time Tp and the position measurement time Tm contravenes the determined condition to be respected.

2. The system according to the claim 1, in which the movement sensor comprises at least one accelerometer which measures an acceleration of the probe with respect to gravitational acceleration, and in which the movement-measuring module is configured to detect a movement of the body when the measured acceleration is greater than or equal to a determined value.

3. The system according to claim 1, in which the probe comprises at least two pressure sensors operating independently of each other, and having distinct detection areas which are not superimposed, and in which the processing module (4) measures a differential pressure in each detection area.

4. The system according to claim 1, in which the processing module is configured to calculate a first difference ΔT1 between a first pressure measurement time Tp1 and a first movement measurement time Tm1,
And in which a first condition to be respected is that the first difference ΔT1 is comprised within a determined time interval.

5. The system according to the claim 4, in which the processing module is configured to calculate a second difference ΔT2 between a second pressure measurement time Tp2, subsequent to the first pressure measurement time Tp1, and a second movement measurement time Tm2, subsequent to the first movement measurement time Tm1,
And in which a second condition to be respected is that the second difference ΔT2 is comprised within a determined time interval.

6. The system according to the claim 5, in which the pressure module is configured to detect the first pressure measurement time Tp1 as being the initiation of a contraction and the second pressure measurement time Tp2 as being the end of said contraction, and in which the movement module is configured to detect the first movement measurement time Tm1 as being the start of a set of movements and the second movement measurement time Tm2 as being the end of said set of movements.

7. The system according to claim 1, in which the probe comprises a first section intended to be placed in a proximal area of the internal canal of the patient and a second section intended to be placed in a distal area of the internal canal when the probe is inserted there, the movement sensor being housed in the second section of the probe, such that a measurement axis corresponds to the axis of a distal area of the internal canal.

8. The system according to claim 7, in which the first section comprises at least one first pressure sensor suitable for measuring the pressure applied by the canal of the patient on the external surface of the body of the probe in a proximal area of the canal and the second section comprises at least one second pressure sensor suitable for measuring the pressure applied by the canal of the patient on the external surface of the body of the probe in a distal area of the canal.

9. The system according to claim 7, in which the probe is elastically flexible such that it can adopt a first position, called the initial position, in which the first section is inclined with respect to the second section by an angle of inclination with a determined value, called the rest value, and a second position, called the curved position, in which the angle of inclination deviates from the rest value by flexibility.

10. The system according to claim 9, in which the value of the angle (α) of inclination at rest is substantially equal to 162°, and in which the value of the angle (α) of inclination when the probe is in the curved position deviates from the value at rest by a maximum of +/−30°.

11. The system according to claim 1, in which the notification device comprises an interface making it possible to visually display data relating to contractions performed by the patient.

12. A method for measuring contractions of the pelvic floor muscles (PFM) using a system according to claim 1, the method comprising the following steps:
measuring at least one differential pressure in the canal of the patient, denoted ΔP, the differential pressure having, as reference pressure, values measured in a relaxed state of the patient in which the pelvic floor muscles are relaxed and associating a measured differential pressure ΔP with a pressure measurement time Tp;
detecting a movement of the patient on the basis of at least one measurement of the movement of the probe and associating the detected movement with a measurement time Tm;
calculating a difference ΔT between the pressure measurement time Tp and the position measurement time Tm and comparing the difference ΔT with a determined condition;
notifying the patient that the synergy between the movement of the body and the contraction of the pelvic floor muscles is suboptimal when the difference between the pressure measurement time Tp and the position measurement time Tm does not respect the determined condition.

13. The system according to claim 8, in which the probe is elastically flexible such that it can adopt a first position, called the initial position, in which the first section is inclined with respect to the second section by an angle of inclination with a determined value, called the rest value, and a second position, called the curved position, in which the angle of inclination deviates from the rest value by flexibility.
